(19) United States
(12) Patent Application Publication
(10) Pub. No.: US 2024/0425875 A1
(43) Pub. Date: Dec. 26, 2024

(54) EXPRESSION SYSTEM FOR CONSTITUTIVE OR CUMATE-INDUCIBLE EXPRESSION IN CHO CELLS

(71) Applicant: National Research Council of Canada, Ottawa (CA)

(72) Inventors: Yves DUROCHER, Montreal (CA); Simon JOUBERT, Laval (CA); Sylvie PERRET, Montreal (CA); Martin LOIGNON, Ste-Anne-de-Bellevue (CA); Jean-Sebastien MALTAIS, Montreal (CA); Simon LORD-DUFOUR, Montreal (CA)

(73) Assignee: National Research Council of Canada, Ottawa, ON (CA)

(21) Appl. No.: 18/691,190

(22) PCT Filed: Sep. 12, 2022
(86) PCT No.: PCT/IB2022/058588

§ 371 Date: Mar. 12, 2024

Related U.S. Application Data
(60) Provisional application No. 63/243,340, filed on Sep. 13, 2021.

(51) Int. Cl. C12N 15/85; C07K 14/21; C12P 21/02

(52) U.S. Cl. CPC C12N 15/85; C07K 14/21; C12P 21/02; C07K 2319/09; C12N 2800/40; C12N 2830/002; C12N 2830/005; C12N 2840/203

(57) ABSTRACT

Provided are methods, expression systems, kits, and vectors for constitutive and/or cumate-inducible expression of a gene of interest in CHO cells. The expression systems and methods described herein employ CHO cells that are stably transfected with a nucleic acid molecule encoding a reverse cumate transactivator (rcTA), the expression of which is regulated by a cymene repressor (CymR). for constitutive and/or cumate-inducible expression of a gene of interest.

### FIELD

The present disclosure relates generally to the field of recombinant expression and, more particularly to the field of recombinant protein expression in mammalian cell lines.

### BACKGROUND

Techniques in molecular and cellular biology often involve the introduction of exogenous genetic material into a cell and the expression of an exogenous sequence within the cell to generate an encoded RNA, polypeptide or protein. A sequence (aka gene of interest) encoding a desired product of interest (aka a gene product of interest), such as an RNA, polypeptide, or protein, is typically incorporated into a suitable vector under the control of a promoter sequence and regulatory elements suitable for the binding of appropriate transcription factors and RNA polymerase in the host cell. In this way, the expression machinery of the host cell is used to co-ordinate expression of the gene product of interest.

Such expression systems may be broadly divided into those that involve constitutive expression, wherein the degree of expression cannot readily be controlled, and those that enable inducible expression, wherein the degree and timing of expression can be controlled by external factors. Inducible expression systems are particularly useful where it is desirable to carefully control expression of a foreign protein in a host cell, for example to simulate a normal, endogenous expression profile or to allow the production of a protein whose constitutive expression might be poorly tolerated, or not tolerated, by the host cell. In some circumstances, the only way to generate a cell line expressing such a protein is to use an inducible expression system, which is maintained in the off state at most times, such that expression may be turned on to a desired level, or for a desired period, at the time that expression is desired.

One example of an inducible expression system is the cumate gene-switch system described in Mullick et al. (2006), U.S. Pat. Nos. 7,745,592, 7,935,788, and 8,728,759. This expression system is derived from the Pseudomonas putida F1 p-cymene operon and uses the regulatory mechanisms of the bacterial operons cmt and cym to regulate gene expression in mammalian cells. Three configurations of the cumate gene switch were described in Mullick et al. (2006): a repressor configuration, a transactivator configuration, and a reverse activator configuration. In the repressor configuration, the cymene repressor (CymR) is used to repress transcription from a constitutive mammalian promoter by binding an operator (CuO) site inserted downstream of the initiation site of the mammalian promoter. Addition of an effector molecule, such as cumate or a cumate analogue, causes CymR to release the DNA, thereby relieving repression and allowing transcription to proceed under the control of the constitutive mammalian promoter. In the activator configuration, CymR is fused to the VP16 activation domain to form a chimeric molecule (cTA) that is used to activate transcription from a minimal CMV promoter placed down-stream of multimerized CuO operators (6×-CuO). The combined 6×-CuO plus minimal CMV promoter is referred to as CR5. In the absence of an effector molecule, cTA binds to CR5, enabling transcription. In the presence of an effector molecule, cTA releases CR5, leading to a loss of transcription. In the reverse activator configuration, a mutant CymR (aka reverse CymR, rCymR) that binds DNA in the presence of an effector molecule, rather than in the absence of an effector molecule, is fused to the VP16 activation domain to produce a chimeric molecule (rcTA) that is used to activate transcription from CR5. In the absence of an effector molecule, rcTA is unable to bind CR5, thus preventing transcription. In the presence of an effector molecule, rcTA is able to bind CR5, thus enabling transcription. The cumate gene-switch was demonstrated by Mullick et al (2006) to allow dose-dependent and tightly controlled expression of reporter molecules in human embryonic kidney 293 (HEK293) cells.

### SUMMARY

The present inventors have developed expression systems, methods, and kits for constitutive and/or cumate-inducible expression of one or more genes of interest in CHO cells.

Accordingly, there is provided a method for constitutive or cumate-inducible expression of a gene of interest in Chinese hamster ovary (CHO) cells, the method comprising:
a. providing a CHO cell line stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA;
b. (i) transfecting cells of the CHO cell line with a vector comprising the gene of interest operably linked to a cumate-responsive promoter and selecting cells that comprise the vector, and
   (ii) culturing the selected cells under conditions that allow the gene of interest to be constitutively expressed, thereby producing a gene product of interest;
c. (i) transfecting cells of the CHO cell line with a first vector comprising the gene of interest operably linked to a cumate-responsive promoter and a second vector comprising a first promoter operably linked to a nucleotide sequence encoding a cymene repressor (CymR), and selecting a cell that comprises the first and second vectors, or
   (i′) transfecting cells of the CHO cell line with a vector comprising the gene of interest operably linked to a cumate-responsive promoter, the vector further comprising a first promoter operably linked to a nucleotide sequence encoding a cymene repressor (CymR), and selecting a cell that comprises the vector, and
   (ii) culturing the selected cell in the presence of an effector molecule under conditions that allow the gene of interest to be expressed, thereby producing the gene product of interest;
d. quantifying the amount of the gene product of interest produced in b) and the amount of the gene product of interest produced in c);
e. comparing the amount of the gene product of interest produced in b) to the amount of the gene product of interest produced in c); and
f. if the amount of the gene product of interest produced in b) is equal to or higher than the amount of the gene product of interest produced in c), selecting constitutive expression for further expression of the gene of interest, or
   if the amount of the gene product of interest produced in b) is lower than the amount of the gene product of interest produced in c), selecting cumate-inducible expression for further expression of the gene of interest.

In an embodiment, the method further comprises:
g. if the amount of the gene product of interest produced in b) is equal to or higher than the amount of the gene product of interest produced in c), repeating steps a) and b) to constitutively produce the gene product of interest, or
   if the amount of the gene product of interest produced in b) is lower than the amount of the gene product of interest produced in c), repeating steps a) and c) to inducibly produce the gene product of interest.

In an embodiment, the method further comprises isolating the gene product of interest and/or purifying the gene product of interest.

In an embodiment of the method, the nucleotide sequence encoding the rcTA has at least 80% sequence identity to the nucleotide sequence set forth in SEQ ID NO: 3. In another embodiment, the nucleotide sequence encoding the rcTA comprises the nucleotide sequence set forth in SEQ ID NO: 3. In another embodiment, the nucleotide sequence encoding the rcTA consists of the nucleotide sequence set forth in SEQ ID NO: 3.

In an embodiment of the method, the first nucleotide sequence further comprises a nucleotide sequence encoding a nuclear localization signal (NLS) linked to the rcTA.

In an embodiment of the method, the CymR response element comprises a plurality of CuO elements. In a further embodiment, the CymR response element comprises (CuO)₂.

In an embodiment of the method, the constitutive promoter is a CMV5 promoter.

In an embodiment of the method, the cumate-responsive promoter is a CR5 promoter.

Another aspect of the disclosure is an expression system for constitutive or cumate-inducible expression of a gene of interest in Chinese hamster ovary (CHO) cells, the expression system comprising:
CHO cells stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA,
a second nucleotide sequence comprising a cumate-responsive promoter and an insertion site to allow insertion of a gene of interest in operable linkage with the cumate-responsive promoter; and
a third nucleotide sequence to enable cumate-inducible expression of the gene of interest, the third nucleotide sequence comprising a first promoter operably linked to a nucleotide sequence encoding a cymene repressor (CymR),
wherein the second nucleotide sequence is comprised by a first vector, the third nucleotide sequence is comprised by a second vector, and the nucleotide sequence encoding the rcTA is codon-optimized for expression in CHO cells.

In an embodiment of the expression system, the nucleotide sequence encoding the rcTA has at least 80% sequence identity to the nucleotide sequence set forth in SEQ ID NO: 3. In an embodiment, the nucleotide sequence encoding the rcTA comprises the nucleotide sequence set forth in SEQ ID NO: 3. In an embodiment, the nucleotide sequence encoding the rcTA consists of the nucleotide sequence set forth in SEQ ID NO: 3.

In an embodiment of the expression system, the first nucleotide sequence further comprises a nucleotide sequence encoding a nuclear localization signal (NLS) linked to the rcTA.

In an embodiment of the expression system, the CymR response element comprises a plurality of CuO elements. In an embodiment, the CymR response element comprises (CuO)₂.

In an embodiment of the expression system, the constitutive promoter is a CMV5 promoter.

In an embodiment of the expression system, the cumate-responsive promoter is a CR5 promoter.

In an embodiment of the expression system, the first promoter is an SV40 promoter.

In an embodiment, the expression system further comprises a gene of interest inserted into the insertion site.

Another aspect of the disclosure is a kit for constitutive or inducible expression of a gene of interest in a Chinese hamster ovary (CHO) cells, the kit comprising:
CHO cells stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA;
a first vector comprising a second nucleotide sequence comprising a cumate-responsive promoter and an insertion site to allow insertion of a gene of interest in operable linkage with the cumate-responsive promoter; and
a second vector comprising a third nucleotide sequence comprising a first promoter operably linked to a nucleotide sequence encoding a cymene repressor (CymR),
wherein the nucleotide sequence encoding the rcTA is codon-optimized for expression in CHO cells.

In an embodiment of the kit, the first nucleotide sequence is as defined in any of paragraphs [0014] to [0021].

In an embodiment of the kit, the first vector is as defined is as defined in any of paragraphs [0014] to [0021].

In an embodiment of the kit, the second vector is as defined is as defined in any of paragraphs [0014] to [0021].

Another aspect of the disclosure is a method for cumate-inducible expression for cumate-inducible expression of a gene of interest in Chinese hamster ovary (CHO) cells, the method comprising:
a providing a CHO cell line stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA; and
b. (i) transfecting cells of the CHO cell line with a first vector comprising the gene of interest operably linked to a cumate-responsive promoter and a second vector comprising a first promoter operably linked to a nucleotide sequence encoding a cymene repressor (CymR), and selecting a cell that comprises the first and second vectors, or
   (i′) transfecting cells of the CHO cell line with a vector comprising the gene of interest operably linked to a cumate-responsive promoter, the vector further comprising a first promoter operably linked to a nucleotide sequence encoding a cymene repressor (CymR), and selecting a cell that comprises the vector, and
c. culturing the selected cell in the presence of an effector molecule under conditions that allow the gene of interest to be expressed, thereby producing the gene product of interest.

In an embodiment, the method further comprises isolating the gene product of interest and/or purifying the gene product of interest.

In an embodiment of the method, the first nucleotide sequence is as defined in any of paragraphs [006] to [0013].

In an embodiment of the method, the first vector is as defined in any of paragraphs [006] to [0013].

In an embodiment of the method, the second vector is as defined in any of paragraphs [006] to [0013].

Another aspect of the disclosure is an expression system for cumate-inducible expression of a gene of interest in Chinese hamster ovary (CHO) cells, the expression system comprising:
CHO cells stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA,
a second nucleotide sequence comprising a cumate-responsive promoter and an insertion site to allow insertion of a gene of interest in operable linkage with the cumate-responsive promoter; and
a third nucleotide sequence comprising a first promoter operably linked to a nucleotide sequence encoding a cymene repressor (CymR),
wherein the second and third nucleotide sequences are comprised by a single vector.

In an embodiment, of the expression system, the vector further comprises a nucleotide sequence encoding a selectable marker to allow for selection of the vector in the CHO cell. In a further embodiment, the nucleotide sequence encoding the selectable marker and the nucleotide sequence encoding the cymene repressor are both operably linked to the first promoter and an internal ribosome entry site (IRES) is positioned between the nucleotide sequence encoding the selectable marker and the nucleotide sequence encoding the cymene repressor. In a still further embodiment, the vector comprises, in order from 5′ to 3′, the first promoter, the nucleotide sequence encoding the selectable marker, the IRES, and the nucleotide sequence encoding CymR.

In an embodiment of the expression system, the first promoter is an SV40 promoter.

In an embodiment of the expression system, the vector further comprises a fourth nucleotide sequence comprising a second cumate-responsive promoter and an insertion site to allow insertion of a second gene of interest in operable linkage with the second cumate-responsive promoter. In a further embodiment, each of the cumate-responsive promoter and the second cumate-responsive promoter is the same promoter. In a still further embodiment, each of the cumate-responsive promoter and the second cumate-responsive promoter is a CR5 promoter.

In an embodiment, the expression system further comprises a gene of interest inserted into the insertion site.

In an embodiment of the expression system, the first nucleotide sequence is as defined in any of paragraphs [0014] to [0021].

In an embodiment of the expression system, the first vector is as defined in any of paragraphs [0014] to [0021].

In an embodiment of the expression system, the second vector is as defined in any of paragraphs [0014] to [0021].

Another aspect of the disclosure is a kit for cumate-inducible expression of a gene of interest in a Chinese hamster ovary (CHO) cell, the kit comprising:
a CHO cell stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA; and
a vector comprising a second nucleotide sequence comprising a cumate-responsive promoter, an insertion site to allow insertion of a gene of interest in operable linkage with the cumate-responsive promoter and comprising a third nucleotide sequence comprising a first promoter operably linked to a nucleotide sequence encoding a cymene repressor (CymR).

In an embodiment of the kit, the first nucleotide sequence is as defined in any of paragraphs [0014] to [0021].

In an embodiment of the kit, the second nucleotide sequence is as defined in any of paragraphs [0014] to [0021].

In an embodiment of the kit, the third nucleotide sequence is as defined in any of paragraphs [0014] to [0021].

Another aspect of the disclosure is an expression vector for cumate-inducible expression in CHO cells, the vector comprising a first nucleotide sequence comprising, in order from 5′ to 3′, a first promoter, a nucleotide sequence encoding a selectable marker, an internal ribosome entry site (IRES), and a nucleotide sequence encoding a cymene repressor (CymR) and a second sequence comprising a cumate-responsive promoter and an insertion site to allow insertion of a gene of interest in operable linkage with the cumate-responsive promoter.

In an embodiment of the expression vector, the first promoter is an SV40 promoter.

In an embodiment of the expression vector, the cumate-responsive promoter is a CR5 promoter.

In an embodiment, the expression vector further comprises a gene of interest inserted into the insertion site.

In an embodiment, the expression vector further comprises a third nucleotide sequence comprising a second cumate-responsive promoter and a second insertion site to allow insertion of a second gene of interest in operable linkage with the second cumate-responsive promoter.

In an embodiment of the expression vector, the second cumate-responsive promoter is a CR5 promoter.

In an embodiment, the expression vector further comprises a second gene of interest inserted into the second insertion site.

### BRIEF DESCRIPTION OF DRAWINGS

FIG. 1 shows a schematic of an embodiment of an expression system according to the disclosure that allows for constitutive expression of a gene of interest: (CuO)ₙ, CymR response element; rcTA, reverse cumate transactivator; Cu. resp. prom., cumate-responsive promoter; GOI, gene of interest; GPOI, gene product of interest.

FIG. 2 shows a schematic of a specific embodiment of an expression system according to the disclosure that allows for constitutive expression of a gene of interest, in which rcTA expression is regulated by a CMV5 promoter and a CymR response element, (CuO)₂, and expression of the gene of interest is regulated by a CR5 promoter.

FIG. 3 shows a schematic of an embodiment of an expression system according to the disclosure that allows for cumate-inducible expression of a gene of interest: CymR, cymene repressor; (CuO)ₙ, CymR response element; rcTA, reverse cumate transactivator; Cu. resp. prom., cumate-responsive promoter; GOI, gene of interest; GPOI, gene product of interest. The upper portion of the figure shows the expression system in the absence of an effector molecule (depicted as cumate) (an “off” state) and the lower portion of the figure shows the expression system in the presence of an effector molecule (depicted as cumate) (an “on” state).

FIG. 4 shows a schematic of a specific embodiment of an expression system according to the disclosure that allows for cumate-inducible expression of a gene of interest, in which CymR expression is regulated by a CMV5 promoter, rcTA expression is regulated by a CMV5 promoter and a CymR response element [(CuO)₂] and expression of the gene of interest is regulated by a CR5 promoter. The upper portion of the figure shows the expression system in the absence of an effector molecule (depicted as cumate) (“off” state) and the lower portion of the figure shows the expression system in the presence of an effector molecule (depicted as cumate) (“on” state).

FIG. 5 shows a schematic of a specific embodiment of an expression system according to the disclosure that allows for cumate-inducible expression of a gene of interest, in which GS and CymR expression is regulated by a pSV40 promoter, rcTA expression is regulated by a CMV5 promoter and a CymR response element [(CuO)₂], and expression of the gene of interest is regulated by a CR5 promoter. The upper portion of the figure shows the expression system in the absence of an effector molecule (depicted as cumate) (“off” state) and the lower portion of the figure shows the expression system in the presence of an effector molecule (depicted as cumate) (“on” state).

FIG. 6 shows a schematic of the pTT®220-PLVZM-LC-HC opt plasmid that was used for cumate-inducible expression of the antibody palivizumab in CHO cells, in accordance with the present disclosure.

FIG. 7 shows the expression level of palivizumab in CHO^{BRI} cells using different versions of rcTA (1A=rcTA, 1B=rcTA codon-optimized for expression in CHO cells, and 1C=codon-optimized rcTA fused to the nuclear localization sequence (NLS) of SV40 large T antigen) under the control of the CMV5 promoter, in the presence (“ON”) and absence (“OFF”) of cumate.

FIG. 8 shows data further evaluating both cTA and rcTA transactivators in CHO cells. Stable pools were generated in CHO cells, expressing either cTA, NLS-cTA, rcTA, or NLS-rcTA using MTX selection. Then, these pools were transfected with plasmids encoding either palivizumab or a palivizumab fusion with the fresno red fluorescent protein, and selected with MSX, before evaluating protein expression in the presence or absence of cumate.

FIG. 9 shows productivity data in either constitutive (C) or cumate-inducible mode (I) of several stable CHO pools, expressing different proteins, that were selected with MSX using CHO²³⁵³™ cell line, using pTT® plasmids that contain or not the CymR expression cassette. FIG. 10 shows productivity data of expression of palivizumab from CR5 promoter following transient transfections, after 7 days of co-expression in different conditions and addition of increasing doses of cumate.

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures, published sequences, and other references mentioned herein are expressly incorporated by reference in their entirety.

### Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

The term “about” as used herein may be used to take into account experimental error, measurement error, and variations that would be expected by a person having ordinary skill in the art. For example, “about” may mean plus or minus 10%, or plus or minus 5%, of the indicated value to which reference is being made.

As used herein the singular forms “a”, “an”, and “the” include plural references unless the context clearly dictates otherwise.

The phrase “and/or”, as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, the term “gene of interest” refers to a DNA sequence encoding a gene product of interest, such as an RNA or polypeptide, for which expression in CHO cells is desired. The gene of interest may be, but need not be, codon-optimized for expression in CHO cells.

As used herein, the term “CymR response element” refers to a nucleic acid molecule that can be bound by CymR and rcTA, and for which the capacity of CymR to bind the CymR response element is dependent on a level of an effector molecule, such as cumate or a cumate analogue. Preferably, the CymR response element comprises one or more cumate operator (CuO) sequences. For example, the CymR response clement may comprise one, two, three, four, five, six, or more CuO operator sequences.

As used herein the term “effector molecule” refers to a molecule that alters the capacity of CymR to bind the CymR response element. The effector molecule may, for example, be cumate, Di-methyl p-aminobenzoic acid (DM PABA), trimethyl cumate, or ethylbenzoate, or a salt thereof. The effector molecule may also be mainly para-or 4-substituted benzoate consisting of a bulky group of heteroatom, such as those selected from the group consisting of 3,4-dimethylbenzoate, 4-ethylbenzoate, 4-t-butylbenzoate, 4-phenylbenzoate, 4-benzylbenzoate, 4-ethoxybenzoate, 4-propyloxybenzoate, 4-n-butyloxybenzoate, 4-chlorobenzoate, 4-bromobenzoate, 4-iodobenzoate, 4-bromomethylbenzoate, 3,4-dichlorobenzoate, 4-trifluoromethylbenzoate, 4-ethyl-m-xylene, 4-vinyltoluene, 4-n-propyltoluene, 4-allytoluene, 4-fluoro-p-toluate, 3-chloro-p-toluate, and 4-bromo-m-toluate. The effector molecule may also be an analogue of cumate such as p-ethylbenzoic acid, p-Propylbenzoic acid, cumic acid, p-isobutylbenzoic acid, p-tert-butylbenzoic acid, p-N-dimethylaminobenzoic acid, or p-N-ethylaminobenzoic acid. In a specific embodiment, the effector molecule is cumate.

As used herein, the term “cumate-inducible”, as used in reference to expression of a gene of interest, means that expression of the gene of interest can be induced by cumate or any other effector molecule that alters the capacity of CymR to bind the CymR response element, for example as described in the preceding paragraph.

As used herein, the term “reverse cumate transactivator” (rcTA) refers to a fusion protein comprising a variant of CymR that binds a CymR response element in the presence of cumate, fused to a VP16 activation domain; as described in Mullick et al 2006 or U.S. Pat. No. 8,728,759.

As used herein, the term “insertion site” refers to any nucleotide sequence that allows for insertion of a gene of interest into a nucleic acid molecule. An insertion site may comprise a contiguous DNA sequence into which a gene of interest may be inserted, for example by recombination, or by enzymatic cleavage followed by ligation. An insertion site may also comprise a pair of DNA ends that are blunt or overhanging, to which a gene of interest may be joined, for example by a ligase or topoisomerase. In a specific embodiment, an insertion site may comprise a multiple cloning site comprising one or more restriction endonuclease recognition sites.

As used herein, the term “cumate-responsive promoter” refers to any promoter that can be bound by CymR and rcTA. In an embodiment, the cumate-responsive promoter comprises a CymR response element positioned 5′ of and operably linked to a minimal promoter. The cumate-responsive promoter may comprise a plurality of CuO sequences operably linked to a minimal CMV promoter. In a specific embodiment, the cumate-responsive promoter comprises six CuO sequences operably linked to a minimal CMV promoter. In a specific embodiment, the cumate-responsive promoter is a CR5 promoter, as described in Mullick et al. (2006).

As used herein, the term “operably linked” and variations thereof, such as “in operable linkage”, when used with respect to a transcription regulatory sequence, such as a promoter or operator sequence, and a nucleotide sequence, such as a gene of interest, in meant to indicate that the regulatory sequence is functionally linked to the nucleotide sequence, such that the regulatory sequence is able to initiate, regulate and/or mediate transcription of the nucleotide sequence. The transcription regulatory sequence and the nucleotide sequence may be directly joined or they may be joined by one or more intervening nucleotides, provided the one or more intervening nucleotides do not prevent the transcription regulatory sequence from initiating, regulating and/or mediating transcription of the nucleotide sequence.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, modified as in Karlin and Altschul, 1993. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present invention. To obtain gapped alignments for comparison purposes, Gapped BLAST can be utilized as described in Altschul et al., 1997. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein in the context of a gene product of interest produced in CHO cells, the term “isolating” refers to a step of releasing the gene product of interest from the CHO cells, for example by lysing the cells or via secretion of the gene product of interest by the cells. Methods for cell lysis are well known in the art and they include, for example, physical disruption, enzymatic disruption, and chemical disruption.

As used herein in the context of a gene product of interest produced in CHO cells, the term “purifying” or “purification” refers to a step of separating the gene product of interest from other components present in the cells and/or from elements present in the growth medium. In order for a gene product of interest to be viewed as “purified”, absolute purity is not required. Rather, the gene product of interest must be separated from a significant portion of components that were present together with the gene product of interest prior to purification. Methods for the purification of gene products are well known in the art. For example, techniques to purify proteins include, but are not limited to, gel filtration chromatography, affinity chromatography, high pressure liquid chromatography (HPLC), electrophoresis, ion exchange chromatography, dialysis, and size fractionation. Techniques to purify RNA include, but are not limited to, phenol-chloroform extraction, silica spin-column absorption, and isopycnic gradient centrifugation.

It should also be understood that, in certain methods described herein that include more than one step or act, the order of the steps or acts of the method is not necessarily limited to the order in which the steps or acts of the method are recited unless the context indicates otherwise.

### Details

The present inventors have created an expression system that can be used for constitutive or cumate-inducible expression of a gene product of interest in CHO cells, using a single CHO cell line for either mode of expression. This expression system allows for rapid identification of the most effective method of expression for any particular gene product of interest, which is difficult to predict and will vary depending on the gene product to be expressed. For any particular gene product, optimal expression conditions may need to be empirically determined, and it is possible that constitutive or inducible expression may be preferable. Inducible expression may be preferred if there is a need to control the timing and/or level of expression, for example if a gene product of interest is toxic to CHO cells. Further, in some cases, inducible expression may lead to higher levels of expression than constitutive expression. However, for some genes of interest, constitutive expression may lead to higher levels of expression than inducible expression, or constitutive and inducible expression may lead to similar levels of expression. In such cases, constitutive expression may be preferred, as it avoids the added cost and complexity associated with inducible expression.

Described herein are nucleic acid molecules comprising various nucleotide sequences and elements thereof, such as transcription regulatory elements and coding elements Nucleotide sequences may be directly joined to other nucleotide sequences, or they may be joined to other nucleotide sequences by one or more intervening nucleotides, provided the intervening nucleotides do not disrupt the function and interoperation of the joined nucleotide sequences. Nucleotide sequences comprised by a nucleic acid molecule, such as a vector, may be directly joined to other identified nucleotide sequences within the nucleic acid molecule or they may be joined by one or more intervening nucleotides. For example, different nucleotide sequences within a vector may be joined by vector backbone sequences or other intervening nucleotide sequences, such as sequences encoding selectable markers or sequences required for replication of the vector.

The relative positioning of nucleotide sequences within a nucleic acid molecule may be described in terms of their order from 5′ to 3′, to indicate the relative order of the identified sequences. When so indicated, the identified sequences are present in the provided order. However, additional non-identified nucleotide sequences and/or intervening nucleotide sequences may also be present. For example, a nucleotide sequence that is described as comprising, in order from 5′ to 3′: a promoter, sequence A and sequence B encompasses a nucleotide sequence that includes, in order from 5′ to 3′: a promoter, sequence A, sequence C, and sequence B.

Functional elements in a fusion polypeptide may be directly joined or they may be joined by a series of one or more amino acid residues, also referred to as a linker; provided the linker does not substantially interfere with the function of the functional elements in the fusion polypeptide. For example, a fusion polypeptide comprising a nuclear localization sequence (NLS) and a reverse cumate transactivator (rcTA) may comprise a linker between the NLS and the rcTA, provided the NLS remains functional to localize the fusion polypeptide to the nucleus and the rcTA retains DNA binding and transactivation activity. Alternately, the NLS may be fused directly to the rcTA, without any additional amino acid(s) being included between the NLS and the rcTA. Linker sequences typically have a length in the range of 1 to 20 amino acids, although longer linkers may be employed, provided the elements of the fusion polypeptide retain function.

An expression system for constitutive or cumate-inducible expression of a gene of interest in Chinese hamster ovary (CHO) cells, in accordance with the present disclosure, may comprise:
CHO cells stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA;
a second nucleotide sequence comprising a cumate-responsive promoter and an insertion site to allow insertion of a gene of interest in operable linkage with the cumate-responsive promoter; and
a third nucleotide sequence to enable cumate-inducible expression of the gene of interest, the third nucleotide sequence comprising a first promoter operably linked to a nucleotide sequence encoding a cymene repressor (CymR),
wherein the second nucleotide sequence is comprised by a first vector, the third nucleotide sequence is comprised by a second vector, and the nucleotide sequence encoding the rcTA is codon-optimized for expression in CHO cells.

An expression system for cumate-inducible expression of a gene of interest in Chinese hamster ovary (CHO) cells, in accordance with the present disclosure, may comprise:
CHO cells stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA),
wherein the constitutive promoter and the CymR response element are operably linked to the nucleotide sequence encoding the rcTA;
a second nucleotide sequence comprising a cumate-responsive promoter and an insertion site to allow insertion of a gene of interest in operable linkage with the cumate-responsive promoter; and
a third nucleotide sequence comprising a first promoter operably linked to a nucleotide sequence encoding a cymene repressor (CymR),
wherein the second and third nucleotide sequences are comprised by a single vector.

An expression vector for cumate-inducible expression in CHO cell, in accordance with the present disclosure, may comprise a first nucleotide sequence comprising, in order from 5′ to 3′, a first promoter, a nucleotide sequence encoding a selectable marker, an internal ribosome entry site (IRES), and a nucleotide sequence encoding a cymene repressor (CymR) and a second sequence comprising a cumate-responsive promoter and an insertion site to allow insertion of a gene of interest in operable linkage with the cumate-responsive promoter.

A kit for constitutive or inducible expression of a gene of interest in a Chinese hamster ovary (CHO) cells, in accordance with the present disclosure, may comprise:
CHO cells stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA;
a first vector comprising a second nucleotide sequence comprising a cumate-responsive promoter and an insertion site to allow insertion of a gene of interest in operable linkage with the cumate-responsive promoter; and
a second vector comprising a third nucleotide sequence comprising a first promoter operably linked to a nucleotide sequence encoding a cymene repressor (CymR),
wherein the nucleotide sequence encoding the rcTA is codon-optimized for expression in CHO cells.

A kit for cumate-inducible expression of a gene of interest in a Chinese hamster ovary (CHO) cell, in accordance with the present disclosure, may comprise:
a CHO cell stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA; and
a vector comprising a second nucleotide sequence comprising a cumate-responsive promoter, an insertion site to allow insertion of a gene of interest in operable linkage with the cumate-responsive promoter and comprising a third nucleotide sequence comprising a first promoter operably linked to a nucleotide sequence encoding a cymene repressor (CymR).

A method for constitutive or cumate-inducible expression of a gene of interest in Chinese hamster ovary (CHO) cells, in accordance with the present disclosure, may comprise:
a. providing a CHO cell line stably transfected with a nucleic acid molecule comprising a first nucleotide sequence, the first nucleotide sequence comprising in order from 540 to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA;
b. (i) transfecting cells of the CHO cell line with a vector comprising the gene of interest operably linked to a cumate-responsive promoter and selecting cells that comprise the vector, and
   (ii) culturing the selected cells under conditions that allow the gene of interest to be constitutively expressed, thereby producing a gene product of interest;
c. (i) transfecting cells of the CHO cell line with a first vector comprising the gene of interest operably linked to a cumate-responsive promoter and a second vector comprising
   a first promoter operably linked to a nucleotide sequence encoding a cymene repressor (CymR), and selecting a cell that comprises the first and second vectors, or
   (i′) transfecting cells of the CHO cell line with a vector comprising the gene of interest operably linked to a cumate-responsive promoter, the vector further comprising a first promoter operably linked to a nucleotide sequence encoding a cymene repressor (CymR), and selecting a cell that comprises the vector, and
   (ii) culturing the selected cell in the presence of an effector molecule under conditions that allow the gene of interest to be expressed, thereby producing the gene product of interest;
d. quantifying the amount of the gene product of interest produced in b) and the amount of the gene product of interest produced in c);
e. comparing the amount of the gene product of interest produced in b) to the amount of the gene product of interest produced in c); and
f. if the amount of the gene product of interest produced in b) is equal to or higher than the amount of the gene product of interest produced in c), selecting constitutive expression for further expression of the gene of interest, or
   if the amount of the gene product of interest produced in b) is lower than the amount of the gene product of interest produced in c), selecting cumate-inducible expression for further expression of the gene of interest.

If the amount of gene product of interest produced by constitutive expression in step b) is equal to or higher than the amount of gene produce of interest produced by cumate-inducible expression in step c), the method may further comprise repeating step b) to constitutively produce the gene product of interest. If the amount of gene product of interest produced by constitutive expression in step b) is less than the amount of gene product of interest produced by cumate-inducible expression in step c), the method may further comprise repeating step c) to inducibly produce the gene product of interest. The amount of gene product produced should be compared on an equivalent basis (for example, by assessing the amount of protein produced under each condition by a particular culture volume).

A method for cumate-inducible expression for cumate-inducible expression of a gene of interest in Chinese hamster ovary (CHO) cells, in accordance with the present disclosure, may comprise:
a. providing a CHO cell line stably transfected with a nucleic acid molecule comprising a first nucleotide sequence, the first nucleotide sequence comprising in order from 5′ to 3′: a constitutive promoter, a CymR response element, and a nucleotide sequence encoding a reverse cumate transactivator (rcTA), wherein the constitutive promoter and the CymR response element are operably linked to the nucleotide sequence encoding the rcTA; and
b. (i) transfecting cells of the CHO cell line with a first vector comprising the gene of interest operably linked to a cumate-responsive promoter and a second vector comprising a first promoter operably linked to a nucleotide sequence encoding a cymene repressor (CymR), and selecting a cell that comprises the first and second vectors, or
   (i′) transfecting cells of the CHO cell line with a vector comprising the gene of interest operably linked to a cumate-responsive promoter, the vector further comprising a first promoter operably linked to a nucleotide sequence encoding a cymene repressor (CymR), and selecting a cell that comprises the vector; and
c. culturing the selected cell in the presence of an effector molecule under conditions that allow the gene of interest to be expressed, thereby producing the gene product of interest.

Methods described herein may further comprise a step of isolating the gene product of interest from the CHO cells using any suitable technique known in the art. Methods described herein may also comprise a step of purifying the gene product of interest using any suitable technique known in the art.

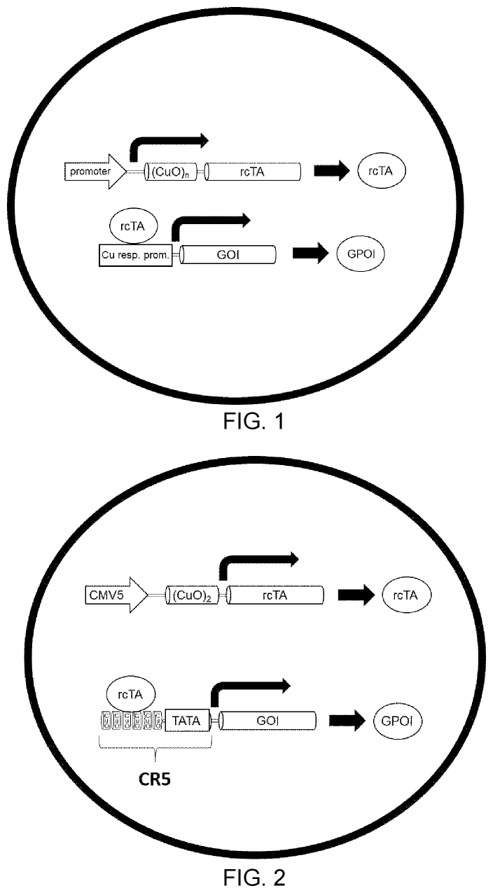

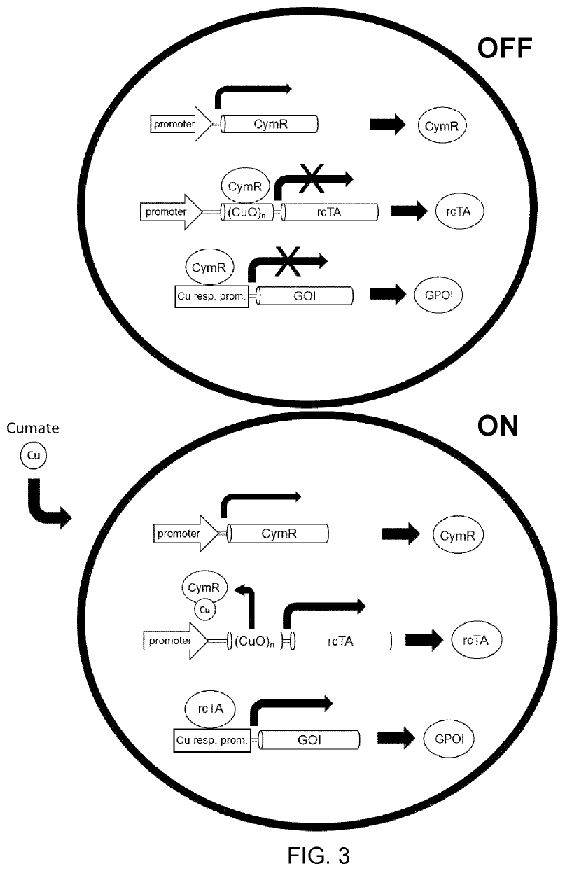

A diagram of an example of an expression system as described herein, and further comprising a gene of interest inserted into the insertion site, is shown in FIG. 1, where the first nucleotide sequence is shown on top and the second nucleotide sequence is shown on bottom. The surrounding oval represents a CHO cell. A specific, non-limiting, embodiment of the expression system is shown in FIG. 2. To aid visualization, the first and second nucleotide sequences are depicted separately, however, they may be comprised by a single nucleic acid molecule or they may be comprised by separate nucleic acid molecules.

If expression of more than one gene product of interest is desired, the expression system may comprise a plurality of cumate-responsive promoters, each with an insertion site to allow insertion of a gene of interest in operable linkage with a respective cumate-responsive promoter. This arrangement may be beneficial if, for example, one wishes to express a protein, such as an antibody or heteromeric protein, that comprises more than one polypeptide. The plurality of cumate-responsive promoters and their respective insertion sites may be comprised by a single nucleic acid molecule or they may be comprised by separate nucleic acid molecules, or any combination thereof (for example, one nucleic acid molecule may comprise a cumate-responsive promoter and insertion site and a second nucleic acid molecule may comprise two cumate-responsive promoters and insertion sites). To allow for production of more than one gene product of interest, the expression system may also comprise a polycistron encoding two or more gene products of interest operably linked to a cumate-responsive promoter.

According to the present disclosure, a constitutive promoter may be any suitable constitutive promoter, natural or engineered, that is functional in CHO cells. Promoters suitable for expression in CHO cells will be known to one skilled in the art, for example as described in Romanova et al. (2017). Examples of suitable constitutive promoters include, but are not limited to, Ubiquitin C (Ubc) promoter, human Elongation Factor 1 alpha (EF1a) promoter, phosphoglycerate kinase 1 (PGK) promoter, simian virus 40 early promoter (SV40) promoter (GenBank accession number J02400.1), cytomegalovirus immediate-early promoter (CMV), chicken b-Actin promoter coupled with CMV early enhancer (CAG), human beta actin promoter, CMV early enhancer (CAG), EF1-HTLV hybrid promoter, and Chinese hamster EF1 promoter (CHEF). In a specific embodiment, the constitutive promoter is a CMV5 promoter.

According to the present disclosure, the first promoter, which regulates expression of CymR, may be any promoter that is able to drive expression in CHO cells, including a constitutive promoter or an inducible promoter. If an inducible promoter is employed, it should be an inducible promoter other than a cumate-responsive promoter. In an embodiment the first promoter is a constitutive promoter, as described herein. In a specific embodiment, the first promoter is a CMV5 promoter or an SV40 promoter.

According to the present disclosure, a CymR response element is a nucleotide sequence comprising one or more cumate operator (CuO) sequences that can be bound by CymR. In an embodiment, the CymR response element comprises one, two, three, or more CuO sequences. In a specific embodiment, the CymR response element comprises two CuO sequences; this configuration may be designated as (CuO)₂.

In some embodiments, the nucleotide sequence encoding rcTA may be codon-optimized for expression in CHO cells. In a further embodiment, the encoded rcTA may be fused to a nuclear localization signal (NLS), such as the SV40 large T-antigen NLS, or any other suitable NLS, as will be known to one skilled in the art. In a specific embodiment, the nucleotide sequence encoding rcTA has at least 80%, at least 85%, at least 90%, at least 91%, at least 92%, at least 93%, at least 94%, at least 95%, at least 96%, at least 97%, at least 98%, or at least 99% identity to the nucleotide sequence set forth in SEQ ID NO: 3. In an embodiment, the nucleotide sequence encoding rcTA comprises or consists of the nucleotide sequence set forth in SEQ ID NO: 3.

According to the present disclosure, a cumate-responsive promoter may be any promoter that can be bound by CymR and rcTA in CHO cells. In an embodiment, the cumate-responsive promoter comprises one or more CuO sequences operably linked to a minimal promoter. In an embodiment, the cumate-responsive promoter comprises a plurality of CuO sequences operably linked to a minimal CMV promoter. In an embodiment, the cumate-responsive promoter comprises six CuO sequences linked to a minimal CMV promoter. In an embodiment, the cumate-responsive promoter is a CR5 promoter, as described in Mullick et al. (2006).

According to the present disclosure, an insertion site may be any nucleotide sequence that allows for insertion of a gene of interest in operable linkage with the cumate-responsive promoter. Any suitable method may be used to insert the gene of interest, and methods for gene insertion will be known to a person skilled in the art, for example as described in Green and Sambrooke (2012). The insertion site may comprise a contiguous DNA sequence into which a gene of interest may be inserted, for example by recombination or by enzymatic cleavage followed by ligation. The insertion site may also comprise DNA ends that are blunt or overhanging, to which a gene of interest may be joined, for example by a ligase or topoisomerase. In an embodiment, a gene of interest is inserted into the insertion site to enable production of a gene product of interest by a CHO cell.

In some embodiments, the first nucleotide sequence may be comprised by a CHO cell. In an embodiment, the first nucleotide sequence is stably maintained by the CHO cell. For example, the first nucleotide sequence may be integrated in the genome of the CHO cell or the first nucleotide sequence may be comprised by an episome that is stably maintained by the CHO cell.

In some embodiments the first nucleotide sequence and/or the second nucleotide sequence may be comprised by a vector. The first and second nucleotide sequences may be comprised by a single vector or they may be comprised by separate vectors, i.e. the expression system may comprise one or more nucleic acid molecules collectively comprising the first nucleotide sequence and the second nucleotide sequence. Any vector(s) suitable for transfection of CHO cells may be employed for use in the expression systems, vectors, methods, and kits as described herein; as will be known to one skilled in the art. In some embodiments, a plasmid or viral vector may be employed. To allow for selection of cells transfected with a vector, the vector may comprise one or more nucleotide sequences encoding selectable marker(s). Examples of suitable selectable markers for use with CHO cells include, but are not limited to, glutamine synthetase, dihydrofolate reductase, blasticidin deaminase, neomycin phosphotransferase, hygromycin B phosphotransferase, zeocin resistance protein, and puromycin N-acetyltransferase. The vector may also comprise a nucleotide sequence encoding a selectable marker, such as an antibiotic resistance cassette, to allow for selection in bacterial cells.

To enable cumate-inducible expression, the expression system may comprise a third nucleotide sequence comprising a first promoter operably linked to a nucleotide sequence encoding a cymene repressor (CymR). The first, second, and third nucleotide sequences may be comprised by a single nucleic acid molecule, two of the nucleotide sequences may be comprised by a single nucleic acid molecule while the other nucleotide sequence is comprised by a separate nucleic acid molecule, or each nucleotide sequence may be comprised by a separate nucleic acid molecule; i.e. expression system may comprise one or more nucleic acid molecules collectively comprising the first nucleotide sequence, the second nucleotide sequence, and the third nucleotide sequence.

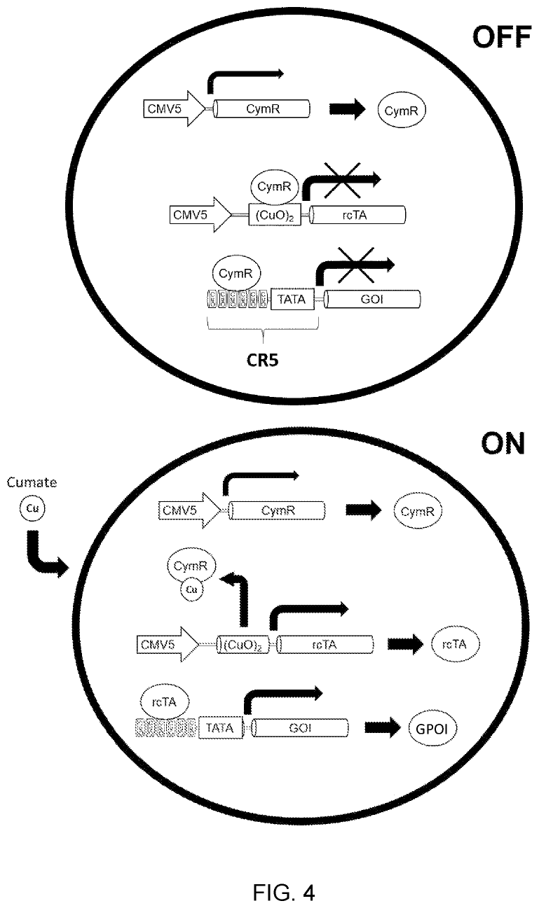

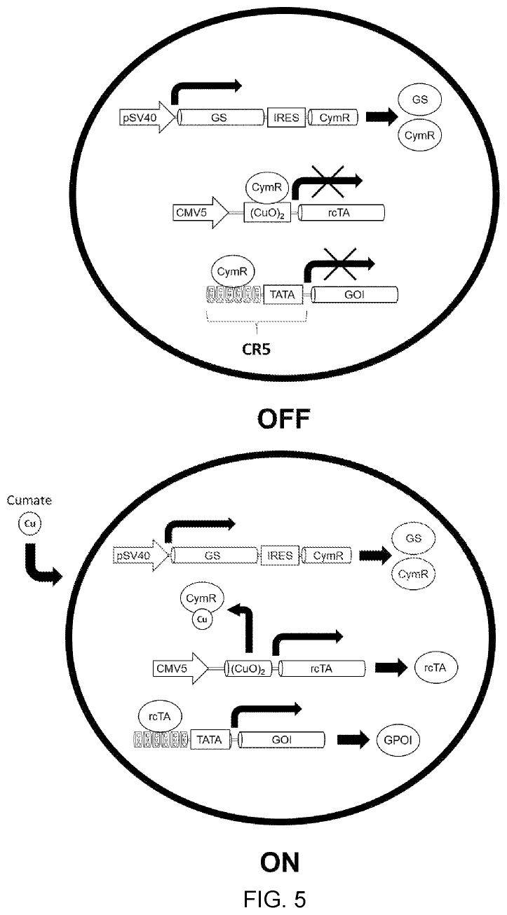

A general diagram of an example of an inducible expression system as described herein, and further comprising a gene of interest inserted into the insertion site, is provided in FIG. 3. A diagram of a specific, non-limiting, example is provided in FIG. 4. In FIGS. 3 and 4, each surrounding oval represents a CHO cell. Within each surrounding oval, the third nucleotide sequence is shown on top, the first nucleotide sequence is shown in the middle, and the second nucleotide sequence is shown on bottom. To aid visualization, the first, second, and third nucleotide sequences are depicted separately, however, they may be comprised by a single nucleic acid molecule, separate nucleic acid molecules, or any combination thereof, as described herein. In FIGS. 3 and 4, the upper image represents the expression system in the “off” state, where an effector molecule (depicted as cumate) has not been provided to the CHO cell and where the cell is cultured under conditions that allow for expression of CymR. The lower image represents the expression system in the “on” state, where an effector molecule (depicted as cumate) has been provided to the CHO cell, causing CymR to release the CymR response element and the cumate-responsive promoter, thereby allowing rcTA to be produced and bind the cumate-responsive promoter, thus activating expression of the gene of interest (GOI) and allowing the gene product of interest (GPOI) to be produced.

In a specific embodiment, the third nucleotide sequence further comprises a nucleotide sequence encoding a nuclear localization signal (NLS) linked in-frame with the nucleotide sequence encoding CymR, such that the encoded polypeptide comprises CymR fused to the NLS. The first promoter may be the same as the constitutive promoter (for example, they may both be CMV5 promoters), or the first promoter may be a different promoter than the constitutive promoter (for example, the constitutive promoter may be a CMV5 promoter and the first promoter may be an SV40 promoter). The constitutive promoter may be any promoter that allows for constitutive expression in CHO cells. The first promoter may be any suitable promoter that allows for expression in CHO cells, including a constitutive promoter or an inducible promoter. If the first promoter is a promoter that allows for inducible expression, it should be inducible by a molecule other than cumate. Suitable constitutive promoters and promoters that allow for inducible expression in CHO cells will be known to one skilled in the art, and examples of suitable promoters are described herein. In a specific embodiment, the first promoter is a CMV5 promoter. In another specific embodiment, the constitutive promoter is a CMV5 promoter and the first promoter is an SV40 promoter.

In some embodiments, the third nucleotide sequence is comprised by a vector. In some embodiments, the first, second, and third nucleotide sequences may be comprised by a single vector, while in other embodiments two of the nucleotide sequences may be comprised by a single vector, while the other nucleotide sequence is comprised by a separate vector. In other embodiments, each of the first, second, and third nucleotide sequences is comprised by a separate vector. In some embodiments, one or two of the nucleotide sequences is/are comprised by a vector, while the other nucleotide sequence(s) is/are comprised by a nucleic acid molecule other than a vector. For example, the other nucleotide sequence(s) may be integrated into the genome of a CHO cell. In an embodiment, the second nucleotide sequence and the third nucleotide sequence are comprised by a single vector that does not comprise the first nucleotide sequence.

In a specific embodiment, the third nucleotide sequence comprises a polycistron operably linked to the first promoter, the polycistron comprising comprising: a nucleotide sequence encoding a selectable marker for selection in CHO cells, an internal ribosome entry sequence (IRES), and the nucleotide sequence encoding CymR. In an embodiment, the third nucleotide sequence comprises, in order from 5′ to 3′, the first promoter, the nucleotide sequence encoding the selectable marker, the IRES, and the nucleotide sequence encoding CymR. In another embodiment, the third nucleotide sequence comprises, in order from 5′ to 3′, the first promoter, the nucleotide sequence encoding CymR, the IRES, and the nucleotide sequence encoding the selectable marker. The selectable marker may be any selectable marker that allows selection in CHO cells, as described herein. In an embodiment, the selectable marker is glutamine synthase (GS). In some embodiments, the nucleotide sequence encoding CymR is joined to a nucleotide sequence encoding an NLS, to allow for production of CymR fused to a NLS. In a specific embodiment, the CymR is codon-optimized for expression in CHO cells.

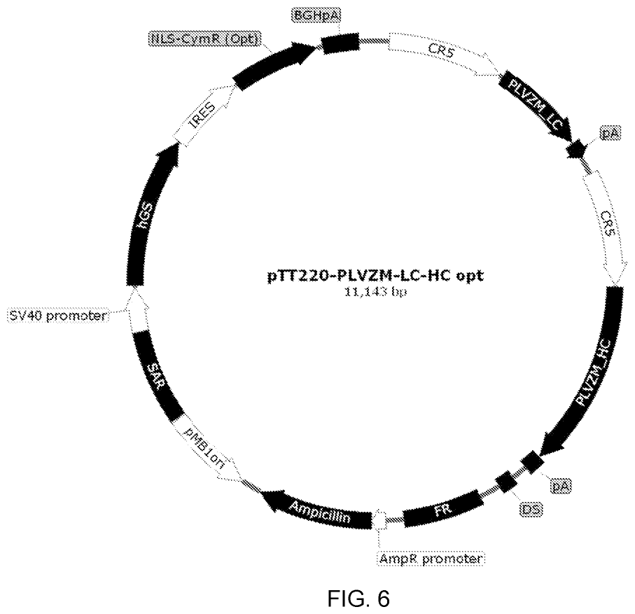

A diagram of a specific, non-limiting, example of such an embodiment, further comprising a gene of interest inserted into the insertion site, is provided in FIG. 5. In FIG. 5, each surrounding oval represents a CHO cell. Within each surrounding oval, the third nucleotide sequence is shown on top, the first nucleotide sequence is shown in the middle, and the second nucleotide sequence is shown on bottom. To aid visualization, the first, second, and third nucleotide sequences are depicted separately, however, they may be comprised by a single nucleic acid molecule, separate nucleic acid molecules, or any combination thereof, as described herein. The upper portion of FIG. 5 represents the expression system in the “off” state, where no effector molecule has been provided to the CHO cell and where the cell is cultured under conditions that allow for expression of CymR. The lower portion of FIG. 5 represents the expression system in the “on” state, where an effector molecule (depicted as cumate) has been provided to the CHO cell, causing CymR to release the CymR response element and the cumate-responsive promoter, thereby allowing rcTA to be produced and bind the cumate-responsive promoter, thus activating expression of the gene of interest (GOI) and allowing the gene product of interest (GPOI) to be produced.

To use an expression system, vector, method, or kit as described herein to produce a gene product of interest, a gene of interest must be inserted into the insertion site of the second nucleotide sequence in operable linkage with the cumate-responsive promoter. To allow constitutive expression, the resulting nucleotide sequence (i.e. the sequence comprising the gene of interest in operable linkage with the cumate-responsive promoter) may then be introduced into a CHO cell stably transfected with the first nucleotide sequence (encoding rcTA). As discussed above, if expression of more than one gene product of interest is desired, the expression system may comprise a plurality of cumate-responsive promoters, each with an insertion site to allow insertion of a gene of interest in operable linkage with a respective cumate-responsive promoter. In this embodiment, an individual gene of interest may be inserted into each insertion site. It is also possible that two or more genes of interest may be present in a polycistron that is inserted into an insertion site.

If cumate-inducible expression is desired, the third nucleotide sequence may also be introduced into the CHO cell. The resulting nucleotide sequence and the third nucleotide sequence may be introduced into the CHO cell simultaneously or they may each be introduced into the CHO cell at different times. Similarly, the resulting and third nucleotide sequences may be comprised by a single nucleic acid molecule, or they may each be comprised by separate nucleic acid molecules. In some embodiments, the expression system may also comprise one or more additional nucleic acid molecules comprising an additional resulting nucleotide sequence, or one or more additional resulting nucleotide sequences may be comprised by the second and/or third nucleic acid molecule(s).

In an embodiment, the second nucleotide sequence and the third nucleotide sequence are comprised by a single vector. To allow for expression of the gene product of interest, the gene of interest is inserted into the vector in operable linkage with the cumate-responsive promoter. The resulting vector, comprising the gene of interest, is then introduced into a CHO cell stably transfected with the first nucleotide sequence (encoding rcTA) to allow for expression of the gene product of interest by the CHO cell under control of rcTA. In an embodiment, the second and third nucleotide sequences are comprised by a single vector, into which the gene of interest is inserted. In another embodiment, the second and third nucleotide sequences are comprised by a single vector, into which the gene of interest is inserted. In some embodiments, the expression system may also comprise one or more additional vectors each comprising an additional resulting nucleotide sequence, or one or more additional resulting nucleotide sequences may be comprised by one of the vectors as described previously in this paragraph.

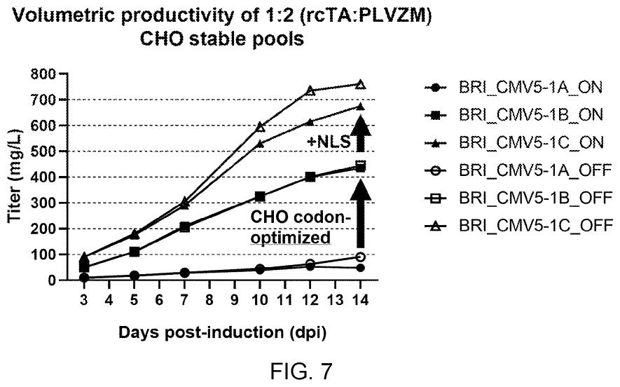

An illustrative example of a plasmid comprising two CR5 promoters, one operably linked to a gene encoding the light chain of palivisumab and the other operably linked to a gene encoding the heavy chain of palivisumab, is provided in FIG. 6. This plasmid further comprises a polycistron comprising GS, an IRES, and NLS-CymR operably linked to an SV40 promoter.

### EXAMPLES

The following non-limiting examples are illustrative of the present disclosure.

### Example 1

### Attempt to use the Cumate Gene-Switch in Reverse Activator Configuration for Inducible Expression in CHO Cells

A plasmid (CMV5-1A) containing the rcTA gene under the control of the constitutive CMV5 promoter was modified to generate two additional versions; CMV5-1B and CMV5-1C. To generate plasmid CMV5-1B, the rcTA coding region was optimized for expression in CHO cells. To generate plasmid CMV5-1C, the nuclear localization sequence of SV40 large T antigen was added at the N-terminus of the codon-optimized rcTA sequence in the CMV5-1B plasmid. The 3 plasmids also contained a DHFR gene under the control of a SV40 promoter to allow selection with methotrexate (MTX).

Naïve CHO cells (CHO^{BRI}) were then transfected with two plasmids at a 1:2 ratio. The first plasmid was either CMV5-1A, 1B, or 1C, and the second plasmid was pTT96-PLVZM which contains the glutamine synthetase gene for selection with MSX, and two CR5 promoters which respectively control expression of the light chain and heavy chain of the antibody palivizumab. Following transfection, stable pools were selected with 125 nM MTX and 62.5 μM MSX. After recovery of cell viability, two fed-batch productions were performed with each stable pool, one supplemented with 2 μg/mL of cumate, and the other not supplemented with cumate.

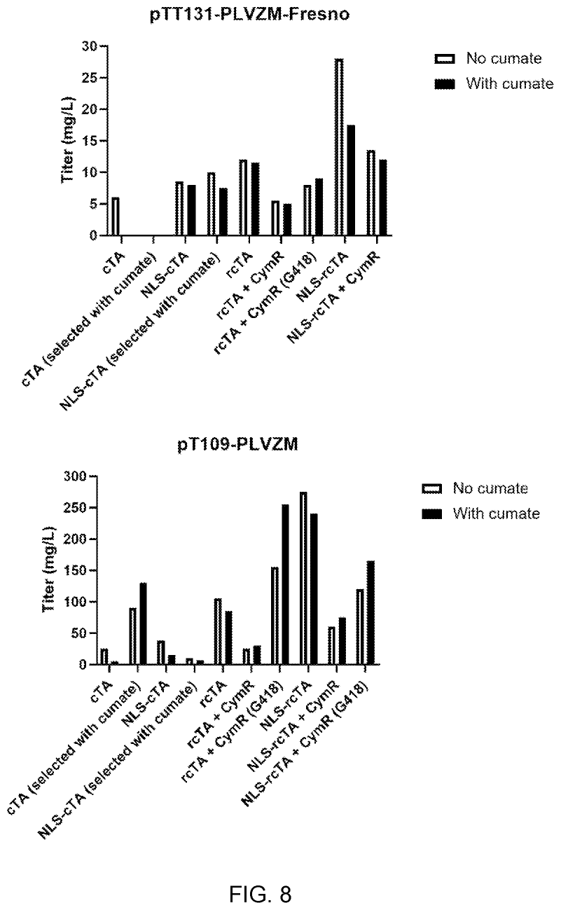

Surprisingly, it was found that productions where no cumate was added produced the antibody to the same level or higher than the productions where cumate was added (see FIG. 7), suggesting that all 3 rcTAs are constitutively active and do not require cumate to bind to and activate the CR5 promoter in CHO cells. In addition, the codon-optimized, NLS-containing rcTA was found to be more efficient than the others at binding and inducing protein production.

In this example, expression of rcTA alone was sufficient to drive protein expression from the CR5 promoter, and this expression was not modulated by cumate. Therefore we found that, contrary to the teaching of Mullick et al (2006), at least in CHO cells, the rcTA can bind and activate the CR5 promoter and is not affected by cumate.

### Example 2

### Evaluation of cTA and rcTA Transactivators in CHO Cells

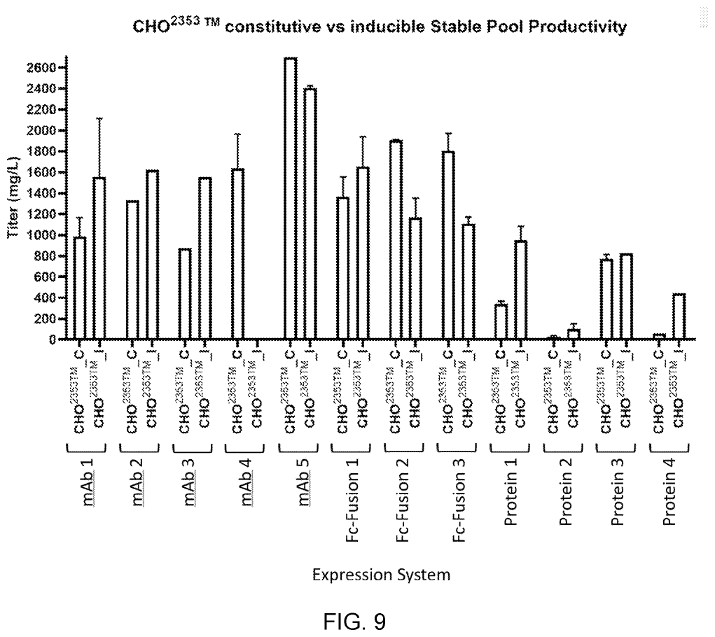

To further evaluate both the cTA and rcTA transactivators in CHO cells and to find which one best drives protein expression, we first generated stable pools in CHO^{BRI} cells, expressing either cTA, NLS-cTA, rcTA, or NLS-rcTA using MTX selection. All four constructs were codon-optimized for expression in CHO cells. The resulting stable pools were then transfected with plasmids encoding either palivizumab or a palivizumab fusion with the fresno red fluorescent protein, and selected with MSX, before evaluating protein expression. In these experiments, the NLS-rcTA stable pools generated the highest palivizumab protein expression, and again, this expression was highest in absence of cumate (see FIG. 8), leading to the conclusion that, in CHO cells, the rcTA transactivator may be a stronger transactivator than the cTA transactivator.

### Example 3

### Determining if rCymR or rcTA Activity can be Modulated by Cumate in CHO Cells

To further evaluate if either rCymR or rcTA activity can be modulated by cumate in CHO cells, the present inventors evaluated expression of palivizumab from the CR5 promoter following transient transfection. After 7 days of co-expression in different conditions and addition of increasing doses of cumate, none of the conditions showed dose-dependent modulation by cumate (FIG. 10). The present inventors therefore concluded that rCymR and rcTA activity is not modulated by cumate in CHO cells, and that rcTA is a constitutively active transactivator of the CR5 promoter (FIG. 2). A 20 μg/mL. dose of cumate was also tested, but it was found to inhibit cell growth.

Since the codon optimized version of NLS-rcTA was found to yield the highest constitutive expression of the transactivators tested, stable CHO cell lines expressing this protein were selected. To carry out this selection, CHO^{BRI} cells were transfected with a plasmid containing the NLS-rcTA under the control of the CMV5_CuO promoter, and a DHFR resistance gene for selection with methotrexate (FIG. 4).

Following transfection of CHO^{BRI} cells, a collection of clones was selected and tested for model protein productivity and for stability. The most productive and stable CHO clone (CHO2353™) was selected from this process. This cell line was tested for constitutive expression of proteins from the CR5 promoter.

### Example 4

### Cumate-Inducible Expression in CHO Cells Using rcTA and CymR

The present inventors further reasoned that, if the CymR repressor was supplied to this cell line, it would be converted into a cumate inducible cell line, since CymR would bind and block the CMV5_CuO promoter, prevent rcTA production, and compete with residual rcTA for binding to the CR5 promoter, thereby blocking expression of the gene of interest. Expression could then be reactivated by adding cumate. The present inventors tested whether we CymR could be supplied using the same plasmid as the plasmid which contains the gene of the protein to be expressed (the gene of interest). To do so, the codon optimized, NLS-CymR was integrated into the plasmid by inserting it after an IRES fused to the GS gene, which expression is driven by the SV40 promoter (FIGS. 5 and 6).

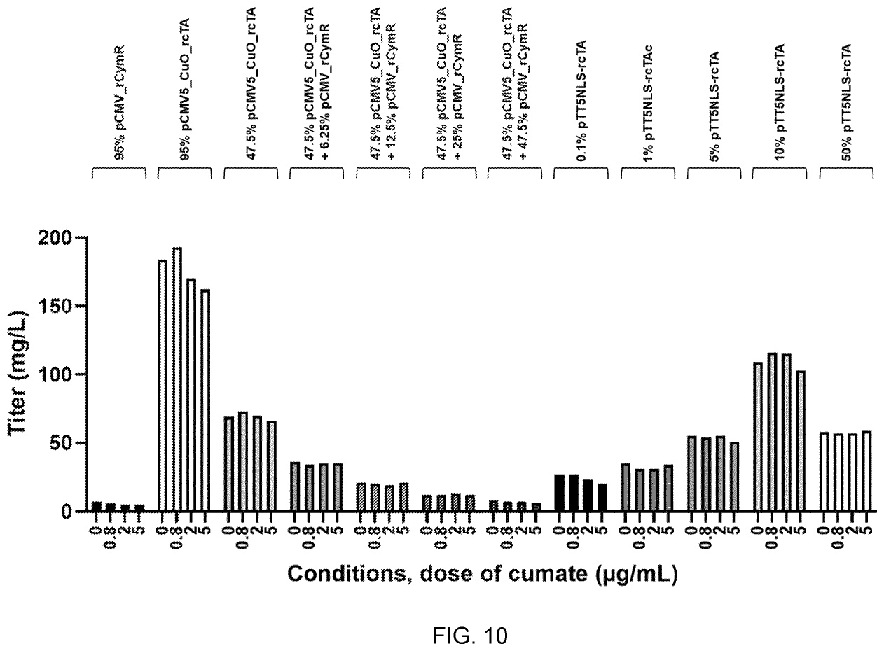

Stable CHO pools were selected with MSX using the CHO2353™ cell line, using pTT® plasmids that contain or not the CymR. pTT®81 (single CR5 promoter cassette for single polypeptide protein expression) and pTT®109 (dual CR5 promoter cassettes for dual polypeptide proteins (for example, monoclonal antibodies)) are control plasmids that do not express CymR, whereas pTT®241 (single CR5 promoter cassette for single polypeptide protein expression) and pTT®220 (dual CR5 promoter cassettes for dual polypeptide proteins (for example, monoclonal antibodies)) arc plasmids that do contain the IRES_CymR region. The present inventors compared stable pool expression in these cell lines for different proteins, and found that: 1) constitutive expression from the CHO^{2353™} cell line was equal to or better than cumate-inducible expression from the CHO55E1™ cell line (data not shown) and 2) for some proteins, cumate-inducible expression from CymR-expressing CHO^{2353™} stable pools was significantly higher than constitutive expression (FIG. 9). Without wishing to be bound by theory, it is believed that there may in some instances be an advantage of placing the CymR within a bicistronic expression cassette. By placing the CymR gene after the IRES sequence, its expression (translation efficiency) is reduced compared to the GS gene. Combined with the use of a weak SV40 promoter driving transcription of the bicistronic cassette, this may allow for generation of pools and clones having a desirable expression level of CymR (higher for “cytotoxic” proteins, lower for “easy-to-express” proteins).

The preceding examples and accompanying drawings are non-limiting and provided to illustrate aspects of the disclosure. As will be apparent to one skilled in the art, specific elements provided in the examples or illustrated in the drawings may be modified, omitted, or substituted, without departing from the disclosure. The invention is defined by the claims, which are to be understood in view of the common general knowledge and the teachings of the disclosure as a whole.

### REFERENCES

The content of each of the following references is hereby incorporated by reference in its entirety.
Green M R and Sambrooke J, Molecular Cloning: A Laboratory Manual (Fourth Edition), Cold Spring Harbour Laboratory Press, 2012, ISBN 978-1-936113-42-2.
Mullick, A., et al., The cumate gene-switch: a system for regulated expression in mammalian cells. BMC Biotechnology, 2006. 6: p. 43.
Romanova N, Noll T. Engineered and Natural Promoters and Chromatin-Modifying Elements for Recombinant Protein Expression in CHO Cells. Biotechnol J. 2018 Mar. 13 (3): e1700232.
U.S. Pat. Nos. 7,745,592, 7,935,788, 8,728,759.

1. A method for constitutive or cumate-inducible expression of a gene of interest in Chinese hamster ovary (CHO) cells, the method comprising:
a. providing a CHO cell line stably transfected with a nucleic acid molecule comprising a first nucleotide sequence, the first nucleotide sequence comprising in order from 5′ to 3′: a constitutive promoter, a cymene repressor (CymR) response element, and a nucleotide sequence encoding a reverse cumate transactivator (rcTA), wherein the constitutive promoter and the CymR response element are operably linked to the nucleotide sequence encoding the rcTA;
b. (i) transfecting cells of the CHO cell line with a vector comprising the gene of interest operably linked to a cumate-responsive promoter and selecting cells that comprise the vector, and
(ii) culturing the selected cells under conditions that allow the gene of interest to be constitutively expressed, thereby producing a gene product of interest;
c. (i) transfecting cells of the CHO cell line with a first vector comprising the gene of interest operably linked to a cumate-responsive promoter and a second vector comprising a first promoter operably linked to a nucleotide sequence encoding a cymene repressor (CymR), and selecting a cell that comprises the first and second vectors, or
(i′) transfecting cells of the CHO cell line with a vector comprising the gene of interest operably linked to a cumate-responsive promoter, the vector further comprising a first promoter operably linked to a nucleotide sequence encoding a cymene repressor (CymR), and selecting a cell that comprises the vector, and
(ii) culturing the selected cell in the presence of an effector molecule under conditions that allow the gene of interest to be expressed, thereby producing the gene product of interest;
d. quantifying the amount of the gene product of interest produced in step b) and the amount of the gene product of interest produced in step c);
e. comparing the amount of the gene product of interest produced in step b) to the amount of the gene product of interest produced in step c); and
f. if the amount of the gene product of interest produced in step b) is equal to or higher than the amount of the gene product of interest produced in step c), selecting constitutive expression for further expression of the gene of interest, or
if the amount of the gene product of interest produced in step b) is lower than the amount of the gene product of interest produced in step c), selecting cumate-inducible expression for further expression of the gene of interest.

2. The method of claim 1, further comprising:
g. if the amount of the gene product of interest produced in step b) is equal to or higher than the amount of the gene product of interest produced in step c), repeating steps a) and b) to constitutively produce the gene product of interest, or
if the amount of the gene product of interest produced in step b) is lower than the amount of the gene product of interest produced in step c), repeating steps a) and c) to inducibly produce the gene product of interest.

3. The method of claim 2, further comprising:
h. isolating the gene product of interest; and/or
i. purifying the gene product of interest.

4. The method of claim 1, wherein the nucleotide sequence encoding the rcTA has at least 80% sequence identity to the full length of the nucleotide sequence set forth in SEQ ID NO: 3.

5-6. (canceled)

7. The method of claim 1, wherein;
the first nucleotide sequence further comprises a nucleotide sequence encoding a nuclear localization signal (NLS) linked to the rcTA;
the CymR response element comprises (CuO)₂:
the constitutive promoter is a CMV5 promoter;
the cumate-responsive promoter is a CR5 promoter; and/or
the first promoter is an SV40 promoter.

8-10. (canceled)

11. An expression system for constitutive or cumate-inducible expression of a gene of interest in Chinese hamster ovary (CHO) cells, the expression system comprising:
CHO cells stably transfected with a nucleic acid molecule comprising a first nucleotide sequence, the first nucleotide sequence comprising in order from 5′ to 3′: a constitutive promoter, a cymene repressor (CymR) response element, and a nucleotide sequence encoding a reverse cumate transactivator (rcTA), wherein the constitutive promoter and the CymR response element are operably linked to the nucleotide sequence encoding the rcTA;
a second nucleotide sequence comprising a cumate-responsive promoter and an insertion site to allow insertion of a gene of interest in operable linkage with the cumate-responsive promoter; and
a third nucleotide sequence to enable cumate-inducible expression of the gene of interest, the third nucleotide sequence comprising a first promoter operably linked to a nucleotide sequence encoding a cymene repressor (CymR),
wherein the second nucleotide sequence is comprised by a first vector, the third nucleotide sequence is comprised by a second vector, and the nucleotide sequence encoding the rcTA is codon-optimized for expression in CHO cells.

12. The expression system of claim 11, wherein the nucleotide sequence encoding the rcTA has at least 80% sequence identity to the full length of the nucleotide sequence set forth in SEQ ID NO: 3.

13-14. (canceled)

15. The expression system of claim 11, wherein;
the first nucleotide sequence further comprises a nucleotide sequence encoding a nuclear localization signal (NLS) linked to the rcTA;
the CymR response element comprises (CuO)₂;
the constitutive promoter is a CMV5 promoter;
the cumate-responsive promoter is a CR5 promoter; and/or
the first promoter is an SV40 promoter.

16-19. (canceled)

20. The expression system of claim 11, further comprising a gene of interest inserted into the insertion site.

21. A kit for constitutive or cumate-inducible expression of a gene of interest in Chinese hamster ovary (CHO) cells, the kit comprising:
CHO cells stably transfected with a nucleic acid molecule comprising a first nucleotide sequence as defined in claim 11;
a first vector as defined in claim 11; and
a second vector as defined in claim 11,
wherein the nucleotide sequence encoding the rcTA is codon-optimized for expression in CHO cells.

22-24. (canceled)

25. A method for cumate-inducible expression of a gene of interest in Chinese hamster ovary (CHO) cells, the method comprising:
a. providing a CHO cell line stably transfected with a nucleic acid molecule comprising a first nucleotide sequence as defined in claim 11; and
b. (i) transfecting cells of the CHO cell line with a first vector as defined in claim 11 and a second vector comprising a first promoter operably linked to a nucleotide sequence encoding a cymene repressor (CymR), and selecting a cell that comprises the first and second vectors, or
(i′) transfecting cells of the CHO cell line with a vector comprising the gene of interest operably linked to a cumate-responsive promoter, the vector further comprising a first promoter operably linked to a nucleotide sequence encoding a cymene repressor (CymR), and selecting a cell that comprises the vector, and
c. culturing the selected cell in the presence of an effector molecule under conditions that allow the gene of interest to be expressed, thereby producing the gene product of interest.

26. The method of claim 25, further comprising:
d. isolating the gene product of interest; and/or
e. purifying the gene product of interest.

27-29. (canceled)

30. An expression system for cumate-inducible expression of a gene of interest in Chinese hamster ovary (CHO) cells, the expression system comprising:
CHO cells stably transfected with a nucleic acid molecule comprising a first nucleotide sequence as defined in claim 11;
a second nucleotide sequence as defined in claim 11; and
a third nucleotide sequence as defined in claim 11,
wherein the second and third nucleotide sequences are comprised by a single vector.

31. (canceled)

32. The expression system of claim 30, wherein the nucleotide sequence encoding the selectable marker and the nucleotide sequence encoding the cymene repressor are both operably linked to the first promoter and an internal ribosome entry site (IRES) is positioned between the nucleotide sequence encoding the selectable marker and the nucleotide sequence encoding the cymene repressor.

33-34. (canceled)

35. The expression system of claim 30, wherein the vector further comprises a fourth nucleotide sequence comprising a second cumate-responsive promoter and an insertion site to allow insertion of a second gene of interest in operable linkage with the second cumate-responsive promoter.

36-37. (canceled)

38. The expression system of claim 30, further comprising a gene of interest inserted into the insertion site.

39-41. (canceled)

42. A kit for cumate-inducible expression of a gene of interest in a Chinese hamster ovary (CHO) cell, the kit comprising:
a CHO cell stably transfected with a nucleic acid molecule comprising a first nucleotide sequence as defined in claim 11;
a vector comprising a second nucleotide sequence as defined in claim 11; and
a nucleic acid molecule comprising a third nucleotide sequence as defined in claim 11.

43-45. (canceled)

46. An expression vector for cumate-inducible expression of a gene of interest in CHO cells, the vector comprising a first nucleotide sequence as defined in claim 11 and a second nucleotide sequence comprising a cumate-responsive promoter and an insertion site to allow insertion of a the gene of interest in operable linkage with the cumate-responsive promoter.

47-48. (canceled)

49. The expression vector of claim 46, further comprising a gene of interest inserted into the insertion site.

50. The expression vector of claim 46, further comprising a third nucleotide sequence comprising a second cumate-responsive promoter and a second insertion site to allow insertion of a second gene of interest in operable linkage with the second cumate-responsive promoter.

51-52. (canceled)


SEQUENCE LISTING (3 sequences)

SEQ ID NO: 1 - DNA, 1071 bp, synthetic construct
  misc_feature 1..1071: note = Nucleic acid encoding an NLS-rcTA fusion protein, codon-optimizedfor expression in CHO cells
  source 1..1071: mol_type = other DNA
atgccaaagaagaagcggaaagtgggtatggtcattatgtcccctaagagaagaactcaggcagagcgtgctatggaaacccagggcaagctgatcgccgctgccctgggcgtgctgagggagaagggctacgctggcttccggatcgccgacgtgcctggagctgctggcgtgagcaggggagctcagtctcaccatttcccaaccaagctggagctgctgctggccacatttgagtggctgtatgagcagatcaccgagagatcccgcgctaggctggctaagctgaagccagaggacgatgtgatccagcagatgctggacgatgctgccgagttctttctggacgatgacttttccatcagcctggacctgatcgtggctgctgaccgcgatcctgtgctgagggagggcatccagcggacagtggagcggaacagattcgtggtgggcgacatctggctgggcgtgctggtgtctcggggcctgtccagagatgacgctgaggatatcctgtggctgatctttaactccgtgagaggcctggccgtgcgcagcctgtggcagaaggataaggagagattcgagagggtgaggaactccaccctggagatcgctcgggagagatacgccaagtttaagagggctgccgctggaggaggaggaggagccgctgccaagctttacagccgcgccaggaccaagaacaattatggctctacaatcgagggcctgctggacctgcctgatgacgatgctccagaggaggctggcctggctgctccaaggctgtccttcctgccagctggacacaccaggcggctgtctaccgctccacctacagacgtgtccctgggcgatgagctgcacctggacggagaggatgtggctatggcccatgctgatgccctggacgatttcgacctggatatgctgggcgacggcgattccccaggaccaggctttacaccccatgactccgccccttatggcgccctggatatggctgactttgagtttgagcagatgtttaccgacgccctggggattgacgagtatggtggttgatga

SEQ ID NO: 2 - protein, 355 aa, synthetic construct
  REGION 1..355: note = NLS-rcTA fusion protein encoded by SEQ ID NO: 1
  REGION 2..8: note = SV40 NLS
  REGION 9..355: note = rcTA
MPKKKRKVGMVIMSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSISLDLIVAADRDPVLREGIQRTVERNRFVVGDIWLGVLVSRGLSRDDAEDILWLIFNSVRGLAVRSLWQKDKERFERVRNSTLEIARERYAKFKRAAAGGGGGAAAKLYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 3 - DNA, 1045 bp, synthetic construct
  misc_feature 1..1045: note = Nucleotide sequence encoding rcTA, codon-optimized for expressionin CHO cells
  source 1..1045: mol_type = other DNA
tatggtcattatgtcccctaagagaagaactcaggcagagcgtgctatggaaacccagggcaagctgatcgccgctgccctgggcgtgctgagggagaagggctacgctggcttccggatcgccgacgtgcctggagctgctggcgtgagcaggggagctcagtctcaccatttcccaaccaagctggagctgctgctggccacatttgagtggctgtatgagcagatcaccgagagatcccgcgctaggctggctaagctgaagccagaggacgatgtgatccagcagatgctggacgatgctgccgagttctttctggacgatgacttttccatcagcctggacctgatcgtggctgctgaccgcgatcctgtgctgagggagggcatccagcggacagtggagcggaacagattcgtggtgggcgacatctggctgggcgtgctggtgtctcggggcctgtccagagatgacgctgaggatatcctgtggctgatctttaactccgtgagaggcctggccgtgcgcagcctgtggcagaaggataaggagagattcgagagggtgaggaactccaccctggagatcgctcgggagagatacgccaagtttaagagggctgccgctggaggaggaggaggagccgctgccaagctttacagccgcgccaggaccaagaacaattatggctctacaatcgagggcctgctggacctgcctgatgacgatgctccagaggaggctggcctggctgctccaaggctgtccttcctgccagctggacacaccaggcggctgtctaccgctccacctacagacgtgtccctgggcgatgagctgcacctggacggagaggatgtggctatggcccatgctgatgccctggacgatttcgacctggatatgctgggcgacggcgattccccaggaccaggctttacaccccatgactccgccccttatggcgccctggatatggctgactttgagtttgagcagatgtttaccgacgccctggggattgacgagtatggtggttgatga